(19) United States
(12) Patent Application Publication
(10) Pub. No.: US 2016/0015630 A1
(43) Pub. Date: Jan. 21, 2016

(54) SPRAYABLE OXYGENATED SALINE COMPOSITION AND METHOD FOR TREATING NASAL CONGESTION, ALLERGY, DRYNESS, EYE IRRITATION, THROAT IRRITATION, WOUNDS, AND SKIN AS APPLIED TO HUMAN TISSUES

(71) Applicant: Oxy Bump Corporation, Miami, FL (US)

(72) Inventor: Shamila Hunter, Miami, FL (US)

(73) Assignee: OXY BUMP CORPORATION, Miami, FL (US)

(21) Appl. No.: 14/800,466

(22) Filed: Jul. 15, 2015

Related U.S. Application Data
(60) Provisional application No. 62/024,750, filed on Jul. 15, 2014.

(51) Int. Cl. A61K 9/00; A61K 36/88; A61K 33/14; A61K 31/417; A61K 36/185; A61K 33/24; A61K 36/64; A61K 36/13; A61K 36/714; A61K 33/42; A61K 33/04; A61K 33/00; A61K 33/18

(52) U.S. Cl. CPC A61K 9/0043; A61K 33/00; A61K 36/88; A61K 9/0053; A61K 9/0048; A61K 9/0014; A61K 33/14; A61K 31/417; A61K 36/185; A61K 33/18; A61K 36/64; A61K 36/13; A61K 36/714; A61K 33/42; A61K 33/04; A61K 33/24

(57) ABSTRACT

An oxygenated saline composition is provided comprising stabilized oxygen, sodium chloride (aqueous saline solution), and optionally one or more homeopathic components. The stabilized oxygen can include quad-atomic oxygen. Also provided is a method for treating, reducing or preventing nasal and/or sinus congestion, allergy, dryness, throat irritation, skin irritation, wounds, or eye irritation comprising administering to an individual in need thereof an effective amount of an oxygenated saline composition comprising stabilized oxygen, aqueous saline solution, and optionally one or more homeopathic components, wherein the composition is administered by intranasal spraying, throat spray, skin spray, sublingually, or by eye drops.

This application claims the benefit of earlier filed U.S. Provisional Application No. 62/024,750, filed on Jul. 15, 2014, which is hereby incorporated by reference herein.

### FIELD OF THE INVENTION

A sprayable product relates to oxygen delivery to the body and benefits to physiological processes. In particular, a sprayable oxygenated saline composition supplements oxygen and other helpful nutrients to the body in the most effective, economic, non-invasive manner.

### BACKGROUND

When the body is in a depleted state from fighting off infection and allergen-created histamines, it is assisted by the delivery of supplemental oxygen. This in turn requires the body to work less to extract oxygen from the allergen-polluted atmosphere or fight to inhale through swollen and/or infected nasal tissues. By not having to work as hard to bring oxygen into the system, the greater residual energy can be dedicated to the body's detoxification and healing processes. The bottom line is that oxygen supplementation in a depleted physiological state may enable a human subject to feel better and recover more quickly.

The administration of oxygen via nasal structures has significant advantages over the intravenous, oral, or transdermal routes. For example, compared to an injection, nasal devices eliminate the pain and the fear associated with the needle. Delivery via the nose also has the following benefits: enhanced patient compliance; decreased needle injury occurrences; no requirement for trained personnel for administration; and decreased the total cost of the treatment; among other advantages described herein.

When compared to the oral route of delivery, nasal devices and delivery systems have other advantages: elimination of gastric and hepatic medication degradation; lower dosage requirements; and importantly, faster onset of action.

Remedies in the cold and allergy market fall into two general categories: over-the-counter (OTC) medications and saline sprays/flushes. OTC products can often create a negative “rebound” congestion caused by overuse. This can create a vicious cycle of overuse and dependence that feels like an addiction.

Rhinitis medicamentosa (RM) is a condition of rebound nasal congestion, brought on by extended use of topical decongestants (e.g., oxymetazoline, phenylephrine, xylometazoline, and naphazoline nasal sprays), that work by constricting blood vessels in the lining of the nose. In as little as 5 days of continued OTC use, onset of RM can begin. Alongside chronic congestion, other symptoms can include regular nose bleeds, headaches, and even a loss of smell and taste. Some habituated or addicted patients also complain of feeling jittery, irritable and thirsty, especially as their medication dose wears off. In very severe cases, oral steroids or nasal surgery may be necessary.

Some saline spray products are spray versus mist and therefore use more product per treatment. The known saline-based products can use as much as 10 ml per treatment. Lesser amounts, i.e. volume is desired for spray treatments administered by the nasal route.

Therefore, if a way could be found to provide an oxygenated saline spray in an efficient manner, this would represent a contribution to the medical and nutraceutical arts. It would also be advantageous to provide an all-natural pH-balanced oxygenated saline spray or mist, optionally containing other beneficial nutrients or minerals, that could be administered as often as desired without any of the adverse effects as discussed.

In view of the above, there is a need and a desire for a stabilized oxygen saline nasal spray in a readily bioavailable formulation.

### SUMMARY OF THE INVENTION

In one embodiment, the invention relates to an oxygenated saline composition comprising stabilized oxygen, sodium chloride (aqueous saline solution), and optionally one or more homeopathic components. The stabilized oxygen can include quad-atomic oxygen.

In another embodiment, an oxygenated saline composition made by a process of: adding sodium chloride to distilled water; adding oxygen (in quad-atomic form) to provide the stabilized oxygen solution; optionally adding one or more homeopathic components to the stabilized oxygen solution; and diluting with water to give the sprayable oxygenated saline composition, is provided.

Also provided is a method for treating, reducing or preventing nasal and/or sinus congestion, allergy, dryness, throat irritation, or eye irritation comprising administering to an individual in need thereof an effective amount of an oxygenated saline composition comprising stabilized oxygen, aqueous saline solution, and optionally one or more homeopathic components, wherein the composition is administered by intranasal spraying.

In one embodiment, a method is provided for treating nasal congestion, allergy, throat, or eye in an individual, including the steps of: providing an oxygenated saline composition comprising stabilized oxygen, aqueous saline solution, and optionally one or more homeopathic components; and administering to the individual in need thereof an effective amount of the oxygenated saline composition; wherein the symptoms of nasal congestion, peak nasal inspiratory flow, allergy, throat irritation, or eye irritation are improved in the individual.

In yet another embodiment, a method is provided for treating damaged human skin or skin wounds in an individual, comprising the steps of: providing an oxygenated saline composition comprising stabilized oxygen, aqueous saline solution, and optionally one or more homeopathic components; and administering to the individual in need thereof an effective amount of the oxygenated saline composition; wherein the damaged skin is improved or skin wounds are healed in the individual.

### BRIEF DESCRIPTION OF THE DRAWINGS

FIG. 1 depicts removal by hand of a lid from atop a spray bottle having a spraying apparatus in one embodiment of the present invention, in which a sprayable oxygenated saline containing composition is provided in a reservoir of the spray bottle.

FIG. 2 depicts insertion of a tip of the spraying apparatus of the spray bottle of FIG. 1 by hand into a nasal cavity of a human subject in an embodiment of the present invention.

FIG. 3 depicts an application of the sprayable oxygenated saline containing composition to the nasal mucosa and/or sinus passages of the nasal cavity by pump compression of the spraying apparatus of FIG. 1 by hand to produce a fine mist or cloud of droplets of/from the composition whereby the composition is efficiently delivered to the target tissues of the subject, i.e., in this instance the nasal cavity.

FIG. 4 depicts average peak nasal inspiratory flow (PNIF) in L/min at various time points (up to 120 minutes) in an embodiment wherein Oxy Bump ‘B’ nasal spray was administered to human subjects compared to baseline.

FIG. 5 depicts average peak nasal inspiratory flow (PNIF) in L/min at various time points (up to 120 minutes) in an embodiment wherein Oxy Bump ‘G’ nasal spray was administered to human subjects compared to baseline.

FIG. 6 depicts average VAS nasal obstruction score at various time points (up to 120 minutes) in an embodiment wherein Oxy Bump ‘B’ nasal spray was administered to human subjects compared to baseline.

FIG. 7 depicts average VAS nasal obstruction score at various time points (up to 120 minutes) in an embodiment wherein Oxy Bump ‘G’ nasal spray was administered to human subjects compared to baseline.

FIG. 8 depicts average VAS rhinorrhea score at various time points (up to 120 minutes) in an embodiment wherein Oxy Bump ‘B’ nasal spray was administered to human subjects compared to baseline.

FIG. 9 depicts average VAS rhinorrhea score at various time points (up to 120 minutes) in an embodiment wherein Oxy Bump ‘G’ nasal spray was administered to human subjects compared to baseline.

FIG. 10 depicts average peak nasal inspiratory flow (PNIF) in L/min at various time points (up to 120 minutes) in an embodiment wherein Oxy Bump ‘T’ throat spray was administered to human subjects compared to baseline.

FIG. 11 depicts average peak nasal inspiratory flow (PNIF) in L/min at various time points (up to 120 minutes) in an embodiment wherein Oxy Bump Eye Drops were administered to human subjects compared to baseline.

FIG. 12 depicts average VAS nasal obstruction score at various time points (up to 120 minutes) in an embodiment wherein Oxy Bump ‘T’ throat spray was administered to human subjects compared to baseline.

FIG. 13 depicts average VAS nasal obstruction score at various time points (up to 120 minutes) in an embodiment wherein Oxy Bump Eye Drops were administered to human subjects compared to baseline.

FIG. 14 depicts average VAS rhinorrhea score at various time points (up to 120 minutes) in an embodiment wherein Oxy Bump ‘T’ throat spray was administered to human subjects compared to baseline.

FIG. 15 depicts average VAS rhinorrhea score at various time points (up to 120 minutes) in an embodiment wherein Oxy Bump Eye Drops were administered to human subjects compared to baseline.

FIG. 16 depicts average VAS score for itching eyes (“itchy eyes”) at various time points (up to 120 minutes) in an embodiment wherein Oxy Bump ‘B’ nasal spray was administered to human subjects compared to baseline.

FIGS. 17-23 depict a scratch/abrasion test showing human subjects treated with the Oxy Bump Skin Spray in an embodiment wherein the treated arm is compared to the untreated arm after 24 hours.

### DETAILED DESCRIPTION

In one aspect, an aqueous composition containing stabilized oxygen solution, sodium chloride (aqueous saline solution), and optionally one or more plant minerals is described. The stabilized oxygen can include quad-atomic oxygen. The plant minerals can include humic and fulvic acids, and other elemental minerals and salts thereof.

This oxygen molecular form makes it very stable and bio-available. Further, due to the intra-nasal delivery system, the oxygenated aqueous composition immediately raises the level of circulating blood oxygen to help increase energy levels and enhance detoxification. Its natural anti-bacterial properties enable the immune system. The intranasal delivery system makes it ideal for fighting cold/flu, allergy, and general nasal congestion and irritation symptoms.

As used herein, the term “tissue” refers to human internal and/or external surfaces, such as, but not limited to, surfaces of the ear, eye, nose, throat, sinuses, and skin

In one aspect, the aqueous composition contains from about 30-35% by weight stabilized oxygen solution, and the balance water (about 65-70%).

In an embodiment, the aqueous composition contains from about 20-25% by weight stabilized oxygen solution, about 1-6% by weight plant minerals in aqueous solution, and the balance water (about 70-74%).

In one preferred embodiment, the aqueous composition contains from about 23.5% by weight stabilized oxygen solution, about 5.8% by weight plant minerals in aqueous solution, and the balance water (about 70.6%).

Various phyto-ionic or plant minerals are useful in the aqueous compositions. In one embodiment plant minerals comprise an aqueous solution containing humic and fulvic acids in a range up to about 57,300 ppm. In another embodiment, humic and fulvic acids can range from about 25,000 to about 75,000 ppm. In yet another embodiment, humic and fulvic acids can range from about 25,000 to about 57,300 ppm.

In a further embodiment plant minerals comprise an aqueous solution containing humic and fulvic acids, and further containing elemental minerals, metals, or salts thereof. The following is a list of representative elemental minerals, one or more of which may be used in the aqueous plant minerals, as shown in Table 1.

TABLE 1

| | Analytical | Per | |
|---|---|---|---|
| Element | measurement | Unit | Test Method |
| Aluminum | 4,626 | ppm | ICP-OES USP <730> |
| Antimony | <0.5 | ppm | ICP-OES USP <730> |
| Barium | <0.5 | ppm | ICP-OES USP <730> |
| Beryllium | 14.80 | ppm | ICP-OES USP <730> |
| Bismuth | 5.354 | ppm | ICP-OES USP <730> |
| Boron | 60.13 | ppm | ICP-OES USP <730> |
| Calcium | 550.8 | ppm | ICP-OES USP <730> |
| Carbon | 180 | ppm | LECO |
| Cerium | 50.614 | ppm | ICP-MS USP <730> |
| Cesium | 0.005 | ppm | ICP-MS USP <730> |
| Chloride | 0.868 | mg/ml | USP <221> Titration |
| Chromium | 6.262 | ppm | ICP-OES USP <730> |
| Cobalt | 19.83 | ppm | ICP-OES USP <730> |
| Copper | 2.633 | ppm | ICP-OES USP <730> |
| Dysprosium | 4.687 | ppm | ICP-MS USP <730> |
| Erbium | 2.525 | ppm | ICP-MS USP <730> |
| Europium | 1.322 | ppm | ICP-MS USP <730> |
| Fluoride | 0.016 | mg/ml | AOAC 939.11 |
| Gadolinium | 6.615 | ppm | ICP-MS USP <730> |
| Gallium | <0.001 | ppm | ICP-MS USP <730> |
| Germanium | 0.088 | ppm | ICP-MS USP <730> |
| Gold | <0.5 | ppm | ICP-MS USP <730> |
| Hafnium | 0.063 | ppm | ICP-MS USP <730> |
| Holmium | 0.908 | ppm | ICP-MS USP <730> |
| Indium | <0.001 | ppm | ICP-MS USP <730> |
| Iodine | 0.094 | mg/ml | ICP-MS USP <730> |
| Iridium | 0.001 | ppm | ICP-MS USP <730> |
| Iron | 20,580 | ppm | ICP-OES USP <730> |
| Lanthanum | 28.19 | ppm | ICP-OES USP <730> |
| Lithium | 4.317 | ppm | ICP-OES USP <730> |
| Lutetium | 0.311 | ppm | ICP-MS USP <730> |
| Magnesium | 2,237 | ppm | ICP-OES USP <730> |
| Manganese | 119.1 | ppm | ICP-OES USP <730> |
| Molybdenum | 0.974 | ppm | ICP-OES USP <730> |
| Neodymium | 30.286 | ppm | ICP-MS USP <730> |
| Nickel | 26.95 | ppm | ICP-OES USP <730> |
| Niobium | <0.5 | ppm | ICP-OES USP <730> |
| Osmium | 0.006 | ppm | ICP-MS USP <730> |
| Palladium | 0.167 | ppm | ICP-MS USP <730> |
| Phosphorus | 691.4 | ppm | ICP-OES USP <730> |
| Platinum | <0.001 | ppm | ICP-MS USP <730> |
| Potassium | 8.200 | ppm | ICP-OES USP <730> |
| Praseodymium | 7.796 | ppm | ICP-MS USP <730> |
| Rhenium | 0.006 | ppm | ICP-MS USP <730> |
| Rhodium | 0.001 | ppm | ICP-MS USP <730> |
| Rubidium | 0.009 | ppm | ICP-MS USP <730> |
| Ruthenium | <0.001 | ppm | ICP-MS USP <730> |
| Samarium | 6.404 | ppm | ICP-MS USP <730> |
| Scandium | 4.877 | ppm | ICP-MS USP <730> |
| Selenium | <0.5 | ppm | ICP-OES USP <730> |
| Silicon | 77.51 | ppm | ICP-OES USP <730> |
| Silver | <0.5 | ppm | ICP-OES USP <730> |
| Sodium | 7.165 | ppm | ICP-OES USP <730> |
| Strontium | 5.524 | ppm | ICP-OES USP <730> |
| Sulfur | 29,925 | ppm | ICP-OES USP <730> |
| Tantalum | 0.012 | ppm | ICP-MS USP <730> |
| Tellurium | <0.5 | ppm | ICP-OES USP <730> |
| Terbium | 0.538 | ppm | ICP-MS USP <730> |
| Thallium | <0.5 | ppm | ICP-OES USP <730> |
| Thorium | 1.963 | ppm | ICP-OES USP <730> |
| Thulium | 0.322 | ppm | ICP-MS USP <730> |
| Tin | <0.5 | ppm | ICP-OES USP <730> |
| Titanium | 0.732 | ppm | ICP-OES USP <730> |
| Tungsten | 4.819 | ppm | ICP-OES USP <730> |
| Vanadium | <0.5 | ppm | ICP-OES USP <730> |
| Ytterbium | 2.057 | ppm | ICP-MS USP <730> |
| Yttrium | 25.61 | ppm | ICP-OES USP <730> |
| Zinc | 94.15 | ppm | ICP-OES USP <730> |
| Zirconium | 2.405 | ppm | ICP-OES USP <730> |
| Mercury | 0.001 | ppm | ICP-MS USP <730> |
| Lead | 0.396 | ppm | ICP-MS USP <730> |
| Arsenic | 3.028 | ppm | ICP-MS USP <730> |
| Cadmium | 0.670 | ppm | ICP-MS USP <730> |
| Nitrogen | <0.2 | % | AOAC 990.03 |
| Total Dissolved Solids | 115,729 | ppm | AOAC 920.193 |

pH: 1.40

In Table 1, the total dissolved solids comprise the elemental minerals and humic and fulvic acids measured at 57,300 ppm.

A sprayable oxygenated saline composition has been discovered. In its principal embodiment, the composition contains stabilized oxygen solution containing sodium chloride. In another embodiment, the composition contains stabilized oxygen solution containing sodium chloride, and one or more plant minerals.

One useful stabilized oxygen solution is Maximum Performance (Aquagen International, Lindon, Utah), of composition: purified water (99.8% by weight), molecules of dissolved oxygen (0.1877%), and sodium chloride (0.00757%); pH 7.5 (av.). The stabilized oxygen solution's active ingredient is a quad-atomic oxygen molecule in a saline suspension. Without being bound by theory, it is believed that the oxygen contained in the stabilized oxygen solution is from a relatively newly identified oxygen molecule known as O₄, i.e., quad-atomic oxygen or “stabilized oxygen.” Cacace, F., et al., Angew. Chem. Intl. Ed. (2001) 40: 4062-4065. The quad-atomic oxygen molecule is safe, stable, and readily absorbed by the body.

In an embodiment, the stabilized oxygen is suspended in a saline solution that is further enriched by a blend of pure plant minerals that enhance the overall health benefits of the product. Useful plant minerals can include natural phyto-ionic materials of other phytonutrients, such as, for example, humic and fulvic acids. Other useful minerals include elemental metals, or salts thereof. Other plant minerals can include, but are not limited to, certain homeopathic components that are useful for treatment of allergy/hay fever, sore throat, earache, and the like.

Alternatively, the oxygenated saline composition contains stabilized oxygen solution containing sodium chloride and at least one homeopathic component.

Representative homeopathic components can include, but are not limited to, the following. Homeopathic dilutions are intended to be exemplary and non-limiting.

For allergy/hay fever, homeopathic components useful for exemplary symptoms in parentheses: Allium Cepa, in alternative dilutions, for example, 6×, 10×, 30×, LM1 HPUS (hay fever, watery eyes); Natrum Muriaticum, 6×, 10×, 30×, LM1 HPUS (sneezing, itchy eyes); Histaminum Hydrochloricum, 10×, 12×, 30×, LM1 HPUS (sinus pain); Quebracho 10×, 30×, LM1 (stimulates respiration, oxygen absorption); Luffa Operculata, 12×HPUS (sneezing, runny nose); and Nux Vomica, 6×HPUS (itchy nose & throat).

For treatment of cold and flu symptoms, exemplary homeopathic components include: Calcarea iodata, 10×, 20×, 30×; Euphrasia officinalis, 10×, 20×, 30×; Quebracho, 10×, 20×, 30×; and Thuja occidentalis, 10×, 20×, 30×.

For sore throat, particularly used in a throat spray, exemplary homeopathic components include: Aconitum napellus, 10×, 20×, 30×, LM1; Ferrum phosphoricum, 10×, 20×, 30×, LM1; Hepar sulphuris calcareum, 10×, 20×, 30×, LM1; Kali bichromicum, 10×, 20×, 30×, LM1; and Quebracho, 10×, 20×, 30×, LM1.

For a sport nasal spray, exemplary homeopathic components include: Quebracho, 10×, 20×, 30×, LM1 (or alternatively, another oxygen/blood supporting ingredient); Adrenalinum, 10×, 20×, 30×, LM1 (enhances adrenal, increases circulation, promotes energy, clearer thinking, well being, deal with stress); Alfalfa, 10×, 20×, 30×, LM1 (improved mental/physical vigor, mental exhilaration); Natrum Carbonic, 10×, 20×, 30×, LM1 (stimulates cellular activity, oxidation, metabolism); Sarcolacticum Acidum, 10×, 20×, 30×, LM1 (relieves acid of muscle exhaustion, stiffness, cramping); Arnica Montana, 10×, 20×, 30×, LM1 (relieves soreness after overexertion); Lycopodium Clavatum, 10×, 20×, 30×, LM1 (circulation, digestive powers); Lactic Acidum, 10×, 20×, 30×, LM1 (lactic acid buildup); Calcarea Carbonica, 10×, 20×, 30×, LM1 (acidity, weakness, swelling, tearing in muscles); Echinacea Purpurea, 10×, 20×, 30×, LM1 (blood corrector, aching, pain); Baryta Carbonic, 10×, 20×, 30×, LM1 (heart function, confidence, weakness, pain, cramping); Lecithin, 10×, 20×, 30×, LM1 (vital processes, favorable upon nutritive condition); and Carbo Vegetabilis, 10×, 20×, 30×, LM1 (oxidation, cramp).

For eye drops, exemplary homeopathic components include: Aconitum napellus, 12×, 20×, 30×; Ferrum phosphoricum, 12×, 20×, 30×; Allium Cepa, 12×, 20×, 30×; Apis mellifica, 12×, 20×, 30×; Cinchona officinalis, 12×, 20×, 30×; Graphites, 12×, 20×, 30×; Rhus toxicodendron, 12×, 20×, 30×; Quebracho, 12×, 20×, 30×; Pulsatilla, 12×, 20×, 30×; and Kali iodatum, 12×, 20×, 30×.

For a baby formula, exemplary homeopathic components include: Quebracho, 12×, 20×, 30×; Thuja occidentalis, 12×, 20×, 30×; and Kali iodatum, 12×, 20×, 30×.

In another embodiment for sore throat, homeopathic components useful for relief of exemplary symptoms in parentheses: Arnica Montana, 3 C HPUS (relieves hoarseness due to overexertion of the vocal cords); Arum triphyllum, 3 C HPUS (relieves vocal cord fatigue in speakers and singers); Belladonna, 3 C HPUS, containing less than 6-10 mg alkaloids per dose (relieves red and sore throat); Bromium, 3 C HPUS (relieves swollen and painful tonsils); Bryonia alba, 3 C HPUS (relieves sore throat with intense thirst); Mercurius solubilis, 3 C HPUS (relieves sore throat with intense salivation); Pulsatilla, 3 C HPUS (relieves raw sore throat); Phytolacca decandra, 3 C HPUS (relieves sore throat with pain spreading to the ears); and Spongia tosta, 3 C HPUS (relieves hoarseness with dry throat).

For earache, homeopathic components useful for exemplary symptoms in parentheses: Pulsatilla, 30 C HPUS (ear pain, worse at night); Chamomilla, 30 C HPUS (restlessness or irritability); Sulphur, 30 C HPUS (ear pain with burning sensation); Calcarea Carbonica, 30 C HPUS (throbbing, pulsing pain in ears); Belladonna, 30 C HPUS (3×10-60% alkaloids)—(fever and inflammation); and Lycopodium, 30 C HPUS (pain and congestion in ears).

An exemplary sprayable oxygenated saline composition is Oxy Bump Saline Oxygen Nasal Spray, available from Oxy Bump Corporation, Miami, Fla.; pH 7-7.4.

The pain of the consumer or patient is the need to find physical relief balanced against the risks of OTC medications or the mess of saline nasal flushing. The sprayable oxygenated saline composition, optionally enriched with one or more homeopathic components, provides the solution. In a preferred embodiment, the composition promotes clean, fast relief that can be sprayed into the nose to contact the nasal and sinus tissues.

When taken or applied by spraying, the sprayable oxygenated saline composition helps to clean the nasal and sinus passages of infectious and irritating materials (both microscopic and larger contaminants). These allergens can remain in the nasal cavities for extended periods of time unless cleared. The spraying of the oxygenated saline composition helps to dislodge them and also can reduce the inflammation caused by these allergens as well.

While most medicated products cause the temporary drying of nasal membranes, the sprayable oxygenated saline composition not only moisturizes, but can help to heal irritated nasal passageways. By facilitating the delivery of an essential life element (namely, oxygen), which enhances the detoxifying release of carbon dioxide, it empowers all of the body's metabolic functions to leave more residual energy to fight off infection. The sprayable oxygenated saline composition accelerates the mending of delicate sinus tissues to create a lasting benefit rather than simple short-term dehydration of these passages.\

By enhancing the delivery of oxygen to the body, cellular respiration and all other physiological processes are stimulated. It is believed that when the oxygenated saline composition is inhaled, the highly bioavailable stabilized oxygen (quad-atomic oxygen) is quickly absorbed from the respiratory system and delivered to the circulatory system. Next, the amount of circulating blood oxygen immediately rises to be delivered on a cellular level.

The use of the sprayable oxygenated saline composition (e.g., Oxy Bump Saline Oxygen Nasal Spray) provides greater relief and healing properties than saline alone and costs less per treatment. Moreover, it is non-addictive, pH balanced and 100% all-natural, and most importantly, has none of the harmful effects of the OTCs.

One important advantage of the sprayable oxygenated saline composition is delivering highly bio-available, pH neutral oxygen solution via the fastest biological delivery system with little degradation of the active ingredient, or discomfort to the consumer, as compared to other methodology.

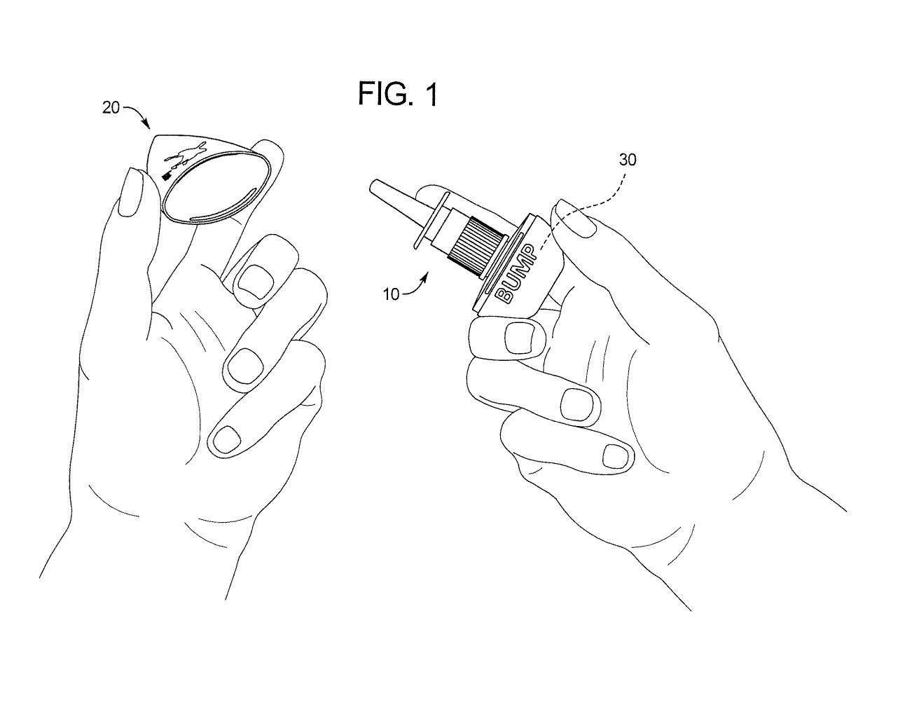

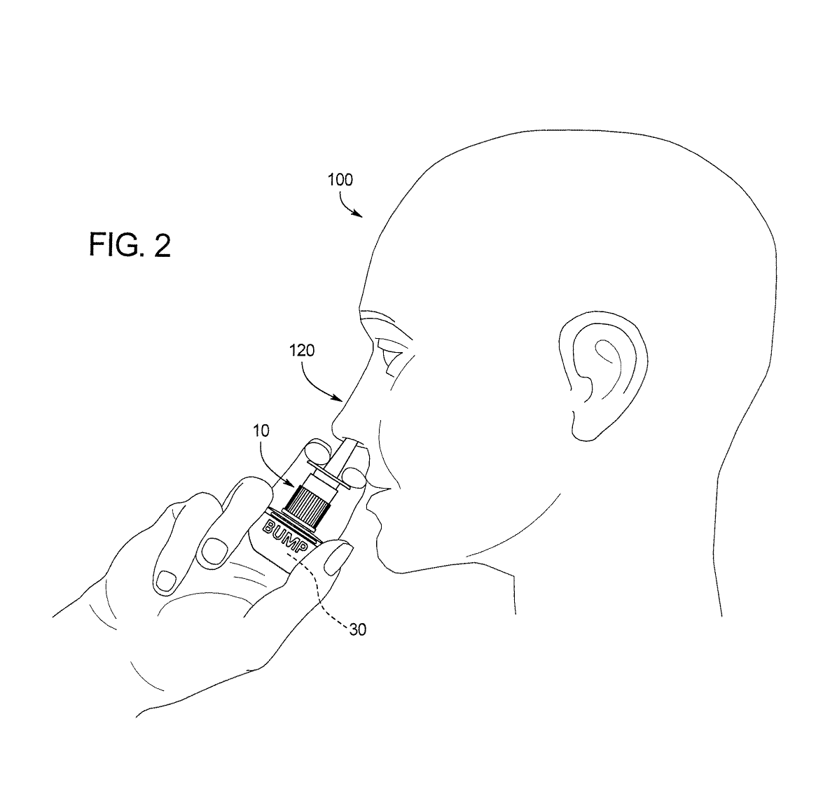

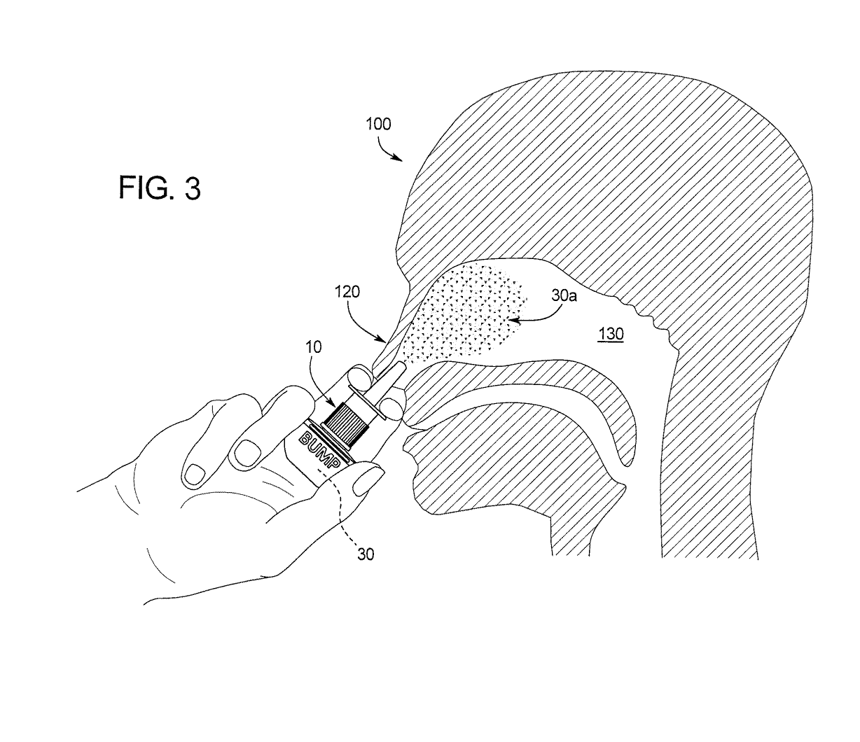

Now turning to the figures, a bottle lid (20) is removed by hand from a spray bottle (10) having an appropriate spraying apparatus which contains a sprayable oxygenated saline containing composition (30) provided in a reservoir of the spray bottle, as shown in FIG. 1. FIG. 2 depicts the insertion of a tip of the spraying apparatus of the spray bottle (10) by hand into a nasal cavity of the nose (120) of a human subject (100) in an embodiment of the present invention. FIG. 3 depicts an application of the sprayable oxygenated saline containing composition to the nasal mucosa and/or sinus passages of the nasal cavity (130) by pump compression of the spraying apparatus of the spray bottle (10) by hand directly into the nose (120) to produce a fine mist or cloud of droplets (30a) of/from the composition whereby the composition is efficiently delivered to the target tissues of the subject (130), i.e., in this instance the nasal cavity.

Delivery System

Intranasal administration is preferred. Useful delivery systems include, but are not limited to, oral or nasal sprays or inhalers, and the like. Other useful delivery systems include, but are not limited to, sublingual, eye drops, ear drops, throat spray, skin spray, and the like.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization, or nebulizer technology.

Also contemplated are moistened towelettes, wipes, wet naps, swabs, and the like, wherein the oxygenated saline composition may be applied by hand. In such a system, the oxygenated saline composition can be provided within a woven or nonwoven cloth or fiber matrix, for example. Administration can be achieved by applying gentle pressure and/or a generally circular wiping motion in order to apply the composition to the skin or other target tissue of a human subject. This application method is generally referred to as swabbing or wiping.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Liquid compositions for oral administration in connection with a method for preventing and/or treating inflammation, colds and/or flu can be prepared in water or other aqueous vehicles. In addition to the above enumerated ingredients or compounds, liquid compositions can include suspending agents such as, for example, methylcellulose, alginates, tragacanth, pectin, kelgin, carrageenan, acacia, polyvinylpyrrolidone, polyvinyl alcohol, and the like. The liquid compositions can be in the form of a solution, emulsion, syrup, gel, or elixir including or containing, together with the above enumerated ingredients or compounds, wetting agents, sweeteners, and coloring and flavoring agents. Various liquid and powder compositions can be prepared by conventional methods. Various ready-to-drink formulations (RTD's) are contemplated.

Homeopathic remedies are made from natural components—mineral, animal, and plant extracts are the base of these natural remedies which are then diluted through altering the degree of concentration to avoid creating side effects that can be disagreeable. Paradoxically, it is postulated that the more a homeopathic remedy is diluted, the more effectively a remedy will work.

There is a very distinct process for making homeopathic remedies. For example, when making a homeopathic remedy which is of plant or animal nature the extract or herb material, which generally includes a soluble substance or substances (or, alternatively, is admixed with another soluble substance), is dissolved in a mixture of alcohol (ethanol) and water (approximately ninety percent pure alcohol and ten percent distilled water, although the ratio can vary). This mixture is optionally set aside for two to four weeks. It is periodically shaken and then is press strained once it has cured. This formulation procedure yields a remedy referred to as a “mother tincture.” See the references that follow, for example, regarding preparation of mother tinctures.

As used herein the terms “homeopathic” and “homeopathic ingredient” are defined in accordance with The Federal Food, Drug and Cosmetic Act (21 U.S.C. 351), which is incorporated herein by reference, and includes those agents or drugs used in the practice of homeopathy and listed in the Homeopathic Pharmacopeia of the United States (HPUS), the entirety of which is also incorporated herein by reference.

Also incorporated herein by reference are the following homeopathic texts: “Materia Medica with Repertory,” 9^{th} Edition, by William Boericke, M.D. (Boericke & Tafel, Santa Rosa, Calif., 1927) and “A Dictionary of Practical Materia Medica,” reprinted 2006, by John Henry Clarke, M.D. (B. Jain Publishers, New Delhi, 2006). Mother tincture examples (as in Clarke): Bryonia alba (white bryony) tincture of root procured before flowering, Vol. I, p. 310; Pulsatilla (windflower herb) tincture of entire fresh plant when in flower, Vol. II, p. 907; Allium Cepa (common red onion) tincture of the onion, or of the whole fresh plant, Vol. I, p. 53.

The mother tincture is diluted to produce different remedy potencies. To do this one may use one of two scales: the decimal (x or “X”) and the centesimal (c or “C”). Optionally, an alcohol/water mixture is used for dilution in various stages.

Between each of the stages of dilution, the diluted tincture is optionally succussed (shaken vigorously). Also, the decimal scale dilution factor (×) is 1:10 and in the centesimal (C) it is 1:100.

For example: To produce a 1 C potency of an Allium cepa remedy, one drop of the mother tincture is added to 99 drops of an alcohol/water mixture and succussed. To produce a 2 C potency, one drop of the 1 C mixture is added to 99 drops of an alcohol/water mixture and succussed. The number of a homeopathic remedy shows how many times it has been diluted and succussed, for example, Allium cepa 6 C has been diluted and succussed six times. Standard dilutions include 6×, 10×, 12×, 20× and 30×.

Without being bound by theory, it is noted that statistical and probability considerations may apply in a given solute/solution combination where dilutions reach or exceed 24×(1/10²⁴ dilution), in comparison with Avogadro's number. The embodiments as described are not intended to be limited merely by theoretical calculations, particularly where a statistical probability may apply.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include citric acid, potassium sorbate, ethanol, aqueous ethanol mixtures, water, fruit and/or vegetable juices, and combinations thereof.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

Routes of Administration

The compositions may be administered by any suitable route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The unique selling property of the sprayable oxygenated saline composition (e.g., Oxy Bump Saline Oxygen Nasal Spray) is the fast delivery of stabilized oxygen via a small, economic, and easy-to-use nasal spray. Due to the intra-nasal delivery system, the sprayable oxygenated saline composition immediately raises the level of circulating blood oxygen to help increase energy levels and enhance detoxification. Also, the sprayable oxygenated saline composition's natural anti-bacterial/antiviral properties can stimulate the immune system. The nasal application makes it ideal for fighting cold/flu, allergy, and general nasal congestion and irritation symptoms. In one embodiment, a small ergonomic bottle can make it easy-to-use and transport so that it can be used anywhere without messy application.

Without limitation, the sprayable oxygenated saline composition can be used to treat, prevent, or help with: healing nasal passages; reducing inflammation, fighting infection, sinus congestion, dry or runny nose, stuffy or swollen nose, cleansing & moisturizing, dry climate relief, headaches, migraines, nasal irritation from foreign matter like dust or pollen, nasal dryness from chromic oxygen gas use, nose bleeds, reduce muscle cramps, poor sleep or insomnia, jetlag, altitude sickness, hangover symptoms, and the like. The sprayable oxygenated saline composition can also be used to promote or help with: breathing easier, providing more energy without jitters, metabolic enhancement, immune boosting, increase mental acuity and brain function, detoxification, enhanced carbon dioxide and toxin release, restoring pH balance, enhance physical performance and endurance, speed recovery from exertion, and the like.

Consequently the use of intranasal delivery in the invention makes a vital difference in the product's effectiveness. Intranasal use of the oxygenated saline composition provides rapid delivery of the active ingredients to the bloodstream and rapid onset of beneficial effects to the user. The ease of use and speed of onset through intranasal delivery provides value to the user and an unexpected synergistic effect. Suitable methods of administration include, but are not limited to, sublingual, buccal, oral, intranasal, inhalational, and the like.

Other types of products containing the oxygenated saline composition and/or uses can include, but are not limited to, throat spray, wound spray, face/skin spray or mist; topical cream ointment, cleansing rinse, eye drops, ear drops, oral rinse, sports nutrition beverages such as waters or ready-to-drink (RTDs), and the like.

In accordance with one embodiment, a method for treatment of nasal congestion is provided, comprising administering to an individual in need thereof a nutraceutically effective amount of an oxygenated saline composition, in optional combination with one or more homeopathic components.

In another embodiment, a nasal spray including an oxygenated saline composition, in optional combination with one or more homeopathic components, for treatment of sinus congestion is contemplated in an advanced formula to relieve head congestion, headache, pressure and achiness in eye regions. Suggested uses are for additional relief of colds, flu or acute hay fever and will also reduce rawness in nose and sinus passages.

In another embodiment, a throat spray or nasal spray including an oxygenated saline composition, in optional combination with one or more homeopathic components, for treatment of cold or flu symptoms is contemplated. These spray embodiments provide powerful immune support and will help fight off cold or flu symptoms. Furthermore, the throat spray will relieve hoarseness, red dry throats and soothe sore, scratchy and inflamed throats.

In yet another embodiment, a skin spray including an oxygenated saline composition, in optional combination with one or more homeopathic components, for treatment of skin damage made by wind, sun, allergens, cuts, scratches, or wounds is contemplated. The skin spray may also be used in conjunction with skin procedures such as, but not limited to, facials, microdermabrasion, peels, or laser treatment.

In accordance with another embodiment, a method for treatment of allergy or allergic inflammation, or other allergic symptoms is provided, comprising administering to an individual in need thereof a nutraceutically effective amount of an oxygenated saline composition.

In accordance with one embodiment, an oxygenated saline composition can be made by a process as follows: adding sodium chloride to distilled water; adding oxygen (in quad-atomic form) to provide the stabilized oxygen solution; optionally adding one or more homeopathic components to the stabilized oxygen solution; and diluting with water to give the sprayable oxygenated saline composition.

In accordance with other embodiments, a method for increasing muscle performance or endurance, or increasing energy and stamina during sports exertion or other strenuous physical activities is provided, comprising administering to an individual in need thereof a nutraceutically effective amount of an oxygenated saline composition.

The aqueous composition and methods described above may be further understood in connection with the following Examples. In addition, the following non-limiting examples are provided to illustrate the invention.

### Example 1A

### Sprayable Oxygenated Saline Formulation

A representative solution of Oxygenated Saline Formulation may include or consist of the following ingredients of Table 2.

TABLE 2

| Ingredient | Weight % |
|---|---|
| Stabilized Oxygen saline solution | 23.5 (g) |
| (AQUAGEN Maximum Performance) | |
| Plant minerals, aq. soln. containing | 5.8 (g) |
| humic/fulvic acids* | |
| Water | 70.6 (g) |

*Optionally including one or more components of Table 1, as discussed above.
pH 7.0-7.4.

### Example 1B

### Process for Preparation

In accordance with one embodiment, food grade sodium chloride is added to distilled water and oxygen is added in to form the stabilized oxygen solution. Plant or phyto-ionic minerals are added as above. The final solution is diluted to the specified volume as in Table 2 for use as a nasal spray formulation.

### Example 1C

In accordance with one embodiment, food grade sodium chloride is added to distilled water and oxygen is added in to form the stabilized oxygen solution. One or more homeopathic dilutions are added as described herein. The final solution is diluted to a specified volume as in Table 5 below for use as a nasal spray formulation.

### Example 2

### Method for treatment of nasal congestion or allergy

In accordance with an embodiment, it is expected that an individual human subject using the oxygenated saline composition of Example 1B by using a dosage of 1-3 sprays into one or both nasal passages, at a dosage per spray of 0.3 mm osculum, 0.1 ml/T, will attain a marked, measurable relief from congestion or stuffiness, within a period of about 10 seconds to about 120 minutes. In a variation, it is expected that an individual will attain a marked, measurable relief from inflammation or other symptoms produced by allergy, within a period of about 10 seconds to about 120 minutes.

### Example 3

### Clinical tests

In accordance with one or more embodiments as described, the compositions of Table 3 were tested using human volunteers.

Study Design

Healthy subjects with a history of nasal congestion in response to pollen, dust mites, cat dander, and/or dog dander, will be enrolled to determine the efficacy of the Oxy Bump study product on attenuating nasal provocation with standardized allergens.

This will be an open-label, adaptive-design study.

Healthy subjects will be screened at visit 1 (V1). Subjects will complete screening for response to allergen exposure, which will provide baseline measurements to compare with active product, as follows.

Subjects will be administered an allergenic challenge consisting of a combination of the following aerosolized allergens to determine the presence or absence of response: Pollen including Tree Pollen (Oak Mix, Birch Mix, Olive Tree, Mountain Cedar, Pecan), Grass Pollen, (Bermuda Grass, KY Bluegrass, Meadow Fescue, Johnson Grass, Perennial Ryegrass); Dust Mites; Cat Dander; Dog Dander.

Subjects will then have their peak nasal inspiratory flow (PNIF) measured multiple times over 1-2-hours as shown in Table 3. PNIF is an objective measure of nasal obstruction during an allergic response, which measures the maximum speed of airflow through the nasal cavity during inspiration and has been shown to be a reliable marker for nasal obstruction. One useful PNIF device is available from Alliance Tech Medical, Granbury, Tex. Subjects will also answer the VAS Nasal Symptom Score multiple times during this hour.

TABLE 3

| V1 | | Time = 0 | | | | Time = |
|---|---|---|---|---|---|---|
| (Screening | | minutes | Time = 15 | Time = 30 | Time = 60 | 120 |
| Visit) | | (Pre- | minutes | minutes | minutes | minutes |
| Study | Time = (−30) | allergen | (post- | (post- | (post- | (post- |
| Schedule | minutes | exposure) | exposure) | exposure) | exposure) | exposure) |
| PNIF | X | | X | X | X | X |
| Allergen | | X | | | | |
| Exposure | | | | | | |
| VAS Nasal | X | | X | X | X | X |
| Symptom | | | | | | |
| Score | | | | | | |

Those subjects who meet all of the inclusion criteria and none of the exclusion criteria will be enrolled and randomized into the study.

The study will enroll n=20 subjects.

Inclusion criteria: healthy volunteers >18 and <65 years of age; subjects who score “moderate” or “severe” on the VAS Nasal Symptom Score in response to an allergenic challenge at screening (V1); and judged by the investigator to be in general good health on the basis of medical history.

Exclusion criteria: pregnant and/or lactating women; subjects who do not meet PNIF criteria at screening; and subjects with idiopathic rhinitis, atrophic rhinitis, or rhinitis medicamentosa.

VAS Subjects will answer a standardized Visual Analog Scale Nasal Symptom Score multiple times after allergen exposure.

For example: VAS NASAL SYMPTOM SCORE is determined at the time points in Table 3.

This scale will be used to evaluate subjects' response to an allergen nasal challenge. The mean score for each subject will determine whether the subject is included in the study. Subjects will be asked to record before and after they have been exposed to the allergen nasal challenge.

The subject is asked to record symptoms on the visual analog scale below with 0 being none, 50 being moderate, and 100 being severe.

VAS symptoms include reporting of: Nasal Obstruction (Stuffy Nose), Rhinorrhea (Runny Nose), Watery Eyes, Itching (Eyes), Itching (Nose), and Sneezing.

Allergen Exposure Protocol (ARM 1 and Optional ARM 2 Efficacy Visits)

Before beginning any assessments, subjects will have been acclimated in the clinic room for one hour in order to allow for a washout of environmental allergens

Subjects will be treated with one dosage of study product thirty (30) minutes prior to allergen exposure. Exemplary dosing: 3 sprays per nostril from a 15 ml spray bottle, approx. 0.35 g-0.4 g/nostril. Following allergen exposure, subjects will have their peak nasal inspiratory flow (PNIF) measured multiple times over two hours. Subjects will also answer the VAS Nasal Symptom Score multiple times during this period. If subjects continue to experience severe symptoms at Time=120 minutes (post-onset of symptoms), subjects will be provided with a rescue dose of medication (50 mg Benadryl).

TABLE 4

| | | Time = 0 | Time = 15 | | | |
|---|---|---|---|---|---|---|
| V2 | | minutes | minutes | | | Time = |
| (Visit) | Time = | (Pre- | (post- | Time = 30 min | Time = 60 min | 120 min |
| Study | (−30) | allergen | alleregen | (post- | (post- | (post- |
| Schedule | minutes | exposure) | exposure) | exposure) | exposure) | exposure) |
| PNIF | X | | X | X | X | X |
| Allergen | | X | | | | |
| Spray | | | | | | |
| Exposure | | | | | | |
| Study | X | | | | | |
| Product | | | | | | |
| Administration | | | | | | |
| VAS | X | | X | X | X | X |
| Nasal | | | | | | |
| Symptom | | | | | | |
| Score | | | | | | |

Arms 3 and 4 were carried out in a similar fashion. Exemplary dosing (Arm 3): 4 sprays down the back of the throat from a 30 ml spray bottle, approx. 0.5 g. Exemplary dosing (Arm 4): 3 drops per eye from a 30 ml dropper bottle, approx. 0.15 g-0.2 g/eye.

As described above, the four arms of the study are carried out as follows.

Study Arms 1-4—Effects of Study Product on Allergy Symptoms

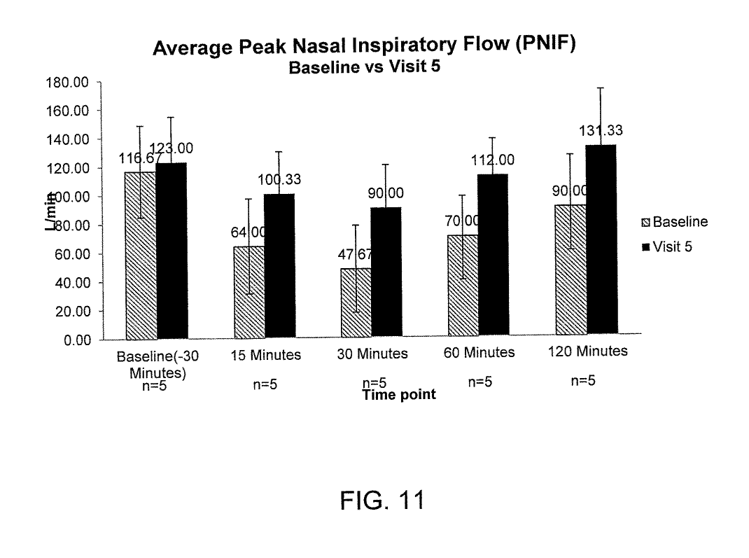

Following a one-week washout, subjects will return to the clinic at V2. At V2, subjects will complete the allergen exposure protocol. Subjects (n=10) will be administered Oxy Bump ‘B’ Nasal Spray (Table 5) immediately after the challenge. After a one-week washout, subjects (n=8) will return to the clinic at V3 and complete the allergen exposure protocol with Oxy Bump ‘G’ Nasal Spray (Table 5). Subjects will complete another one-week washout, subjects (n=7) will return to the clinic at V4 and complete the allergen exposure protocol with Oxy Bump ‘T’ Throat Spray (Table 5). Subjects will complete an additional washout for one week they will return to the clinic at V5 to complete the allergen exposure protocol (n=5) with Oxy Bump Eye Drops (FIGS. 11, 13 and 15).

For example, Oxy Bump ‘B’ or Oxy Bump ‘G’ nasal sprays (See, Table 5) may be applied using 2-3 sprays per nostril from a 15 ml bottle containing 120 sprays, 2×/day, or alternatively, as often as needed. Preferred dosage ranges for the nasal spray can range from about 0.2 g to about 2.0 g per day (total for both nostrils).

For example, Oxy Bump ‘T’ throat spray′ (See, Table 5) may be applied using 3-4 sprays into the back of the throat from a 30 ml bottle containing 240 sprays, 2×/day, or alternatively, as often as needed. Preferred dosage ranges for the throat spray can range about 0.2 g to about 2.0 g per day.

For example, Oxy Bump Eye Drops may be applied using 2-3 drops per eye from a 30 ml bottle containing about 600 drops, 2×/day, or alternatively, as often as needed. Preferred dosage ranges for the eye drops can range about 0.1 g to about 0.6 g per day (total for both eyes).

TABLE 5

| Product designation | | | |
|---|---|---|---|
| (dosage form): | Ingredients | Wt. (lbs) | Weight % |
| OxyBump ‘B’ | Stabilized Oxygen | 0.0109375 | 35% |
| (nasal spray) | saline solution | | |
| | (AQUAGEN | | |
| | Maximum | | |
| | Performance) | | |
| | Deionized (DI) Water | 0.0203125 | 65% |
| | Total | 0.03125 | 100% |
| | | (=15 ml) | |
| OxyBump ‘G’ | AQUAGEN Maximum | 0.0078125 | 25% |
| (nasal spray) | Performance | | |
| | Allergy homeopathic | 0.00625 | 20% |
| | blend* | | |
| | DI water | 0.0171875 | 55% |
| | Total | 0.03125 | 100% |
| | | (=15 ml) | |
| OxyBump ‘Y’ | AQUAGEN Maximum | 0.0078125 | 25% |
| (nasal spray) | Performance | | |
| | Cold/flu homeopathic | 0.00625 | 20% |
| | blend** | | |
| | DI water | 0.0171875 | 55% |
| | Total | 0.03125 | 100% |
| | | (=15 ml) | |
| OxyBump ‘T’ | AQUAGEN Maximum | 0.015625 | 25% |
| (throat spray) | Performance | | |
| | Throat homeopathic | 0.0125 | 20% |
| | blend*** | | |
| | DI water | 0.034375 | 55% |
| | Total | 0.0625 | 100% |
| | | (=30 ml) | |

*Allergy homeopathic blend: equal volumes of Allium Cepa, 10X; Natrum Muriaticum, 10X; Histaminum Hydrochloricum, 10X; and Quebracho 10X.
**Cold/flu homeopathic blend: equal volumes of Calcarea iodata, 10X; Euphrasia officinalis, 10X; Quebracho, 10X; and Thuja occidentalis, 10X.
***Throat homeopathic blend: Aconitum napellus, 10X; Ferrum phosphoricum, 10X; Hepar sulphuris calcareum, 10X; Kali bichromicum, 10X; and Quebracho, 10X.

Oxy Bump Eye Drops contain Stabilized Oxygen saline solution (AQUAGEN Maximum Performance), 20% by weight and balance water.

Clinical Results

AUC Peak Nasal Inspiratory Flow (PNIF)—test of between subject effects of baseline vs. ARM 1 and ARM 2 (V2 and V3).

Percent Change (Other timepoint versus Baseline):

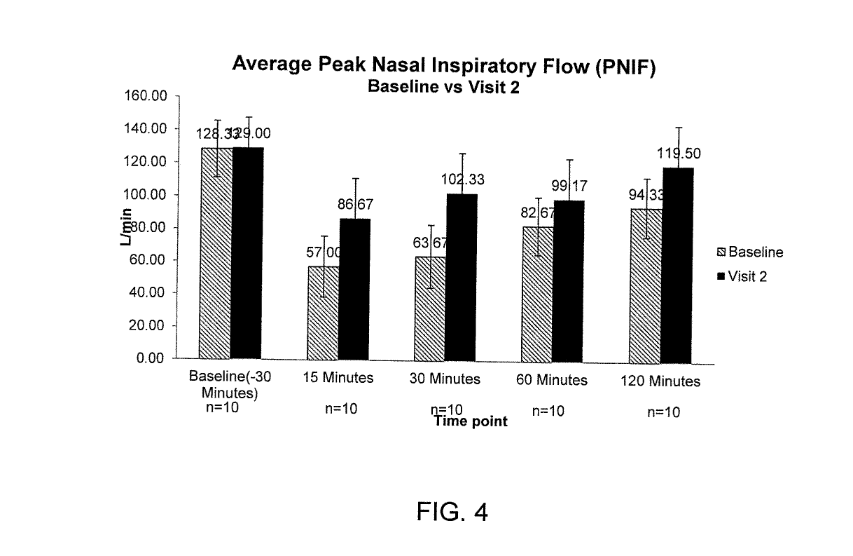

V2=30.13% (sig. 0.013). See FIG. 4.

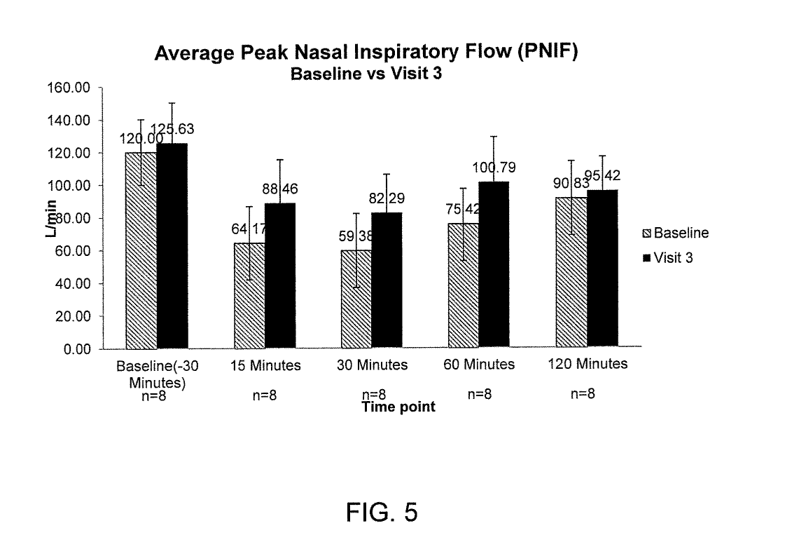

V3=24.33% (sig. 0.061). See FIG. 5.

Note: Significance testing was performed using Paired t-test. Significant at alpha=0.05.

As shown in FIGS. 4 and 5, PNIF (L/min) post-challenge was improved dramatically in human subjects after treatment with Oxy Bump ‘B’ and Oxy Bump ‘G’ compared to baseline.

AUC nasal obstruction (VAS Nasal Symptom Score)—test of between subject effects of baseline vs. ARM 1 and ARM 2 (V2 and V3).

Percent Change (Other timepoint versus Baseline):

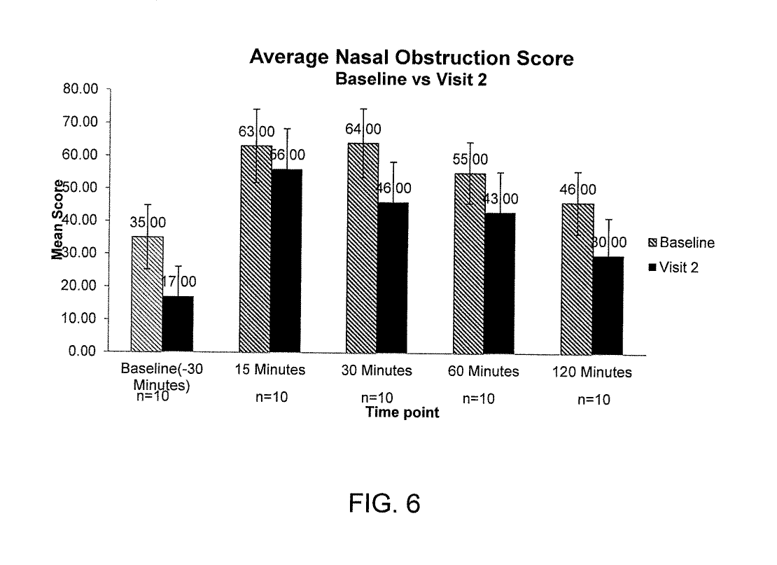

V2=−24.78% (sig. 0.024). See FIG. 6.

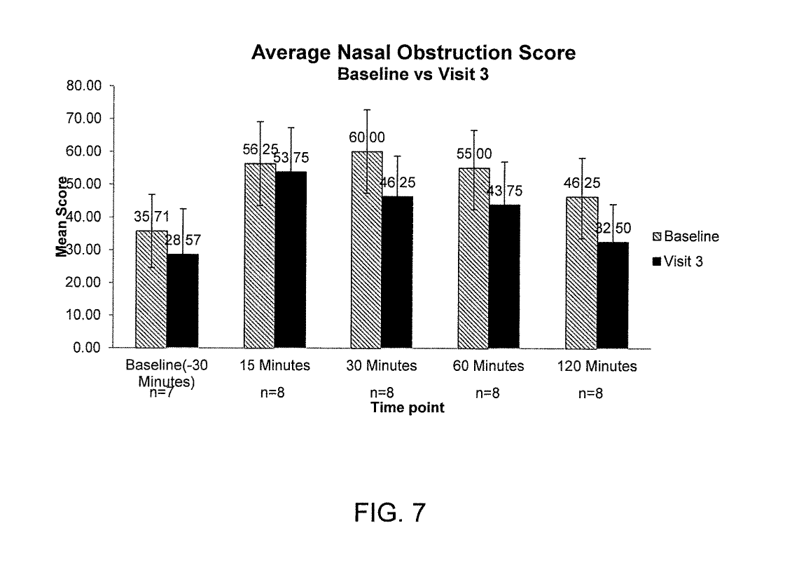

V3=−19.44% (sig. 0.014). See FIG. 7.

Note: Significance testing was performed using Paired t-test. Significant at alpha=0.05.

As shown in FIGS. 6 and 7, VAS nasal obstruction score post-challenge was improved dramatically after treatment with Oxy Bump ‘B’ and Oxy Bump ‘G’ compared to baseline.

AUC rhinorrhea (VAS Nasal Symptom Score)—test of between subject effects of baseline vs ARM 1 and ARM 2 (V2 and V3).

Percent Change (Other timepoint versus Baseline):

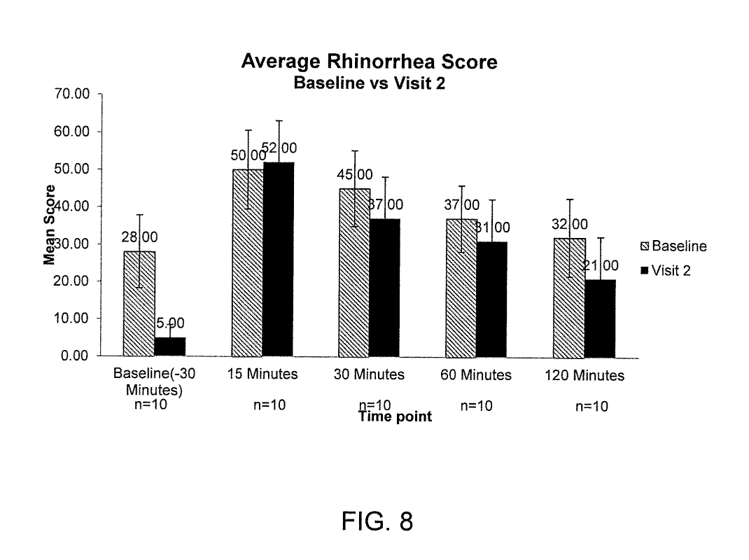

V2=−18.62% (sig. 0.044). See FIG. 8.

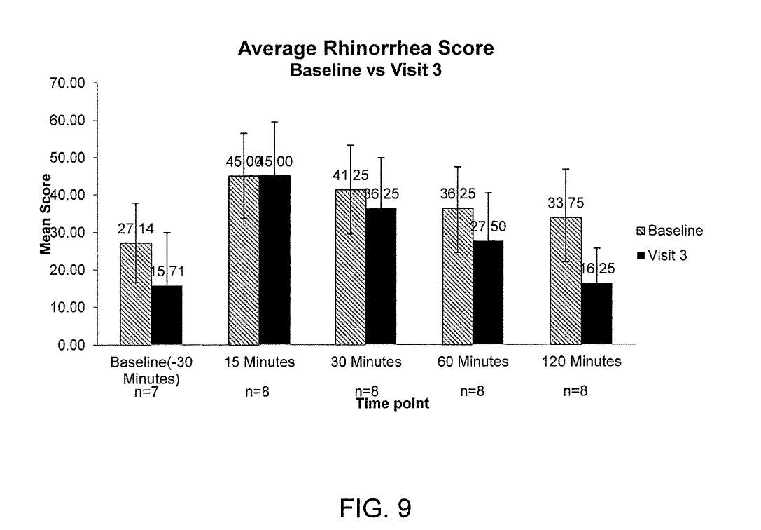

V3=−21.58% (sig. 0.087). See FIG. 9.

Note: Significance testing was performed using Paired t-test. Significant at alpha=0.05.

As shown in FIGS. 8 and 9, VAS rhinorrhea score post-challenge was improved dramatically after treatment with Oxy Bump ‘B’ and Oxy Bump ‘G’ compared to baseline.

AUC itching eyes (VAS Itchy Eyes Symptom Score)—test of between subject effects of baseline vs ARM 1 and ARM 2 (V2 and V3).

Percent Change (Other timepoint versus Baseline):

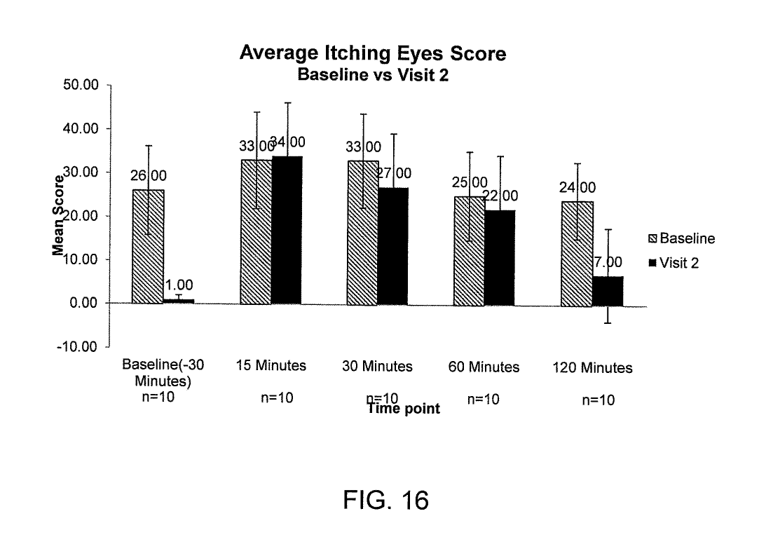

V2=−27.01% (sig. 0.039). See FIG. 16.

V3=−6.32% (sig. 1.000).

Note: Significance testing was performed using Sign Test. Significant at alpha=0.05.

As shown in FIG. 16, VAS itching eyes (“itchy eyes”) score post-challenge was improved dramatically after treatment with Oxy Bump ‘B’ compared to baseline.

AUC Peak Nasal Inspiratory Flow (PNIF)—test of between subject effects of baseline vs. ARM 3 and ARM 4 (V4 and V5).

Percent Change (Other timepoint versus Baseline):

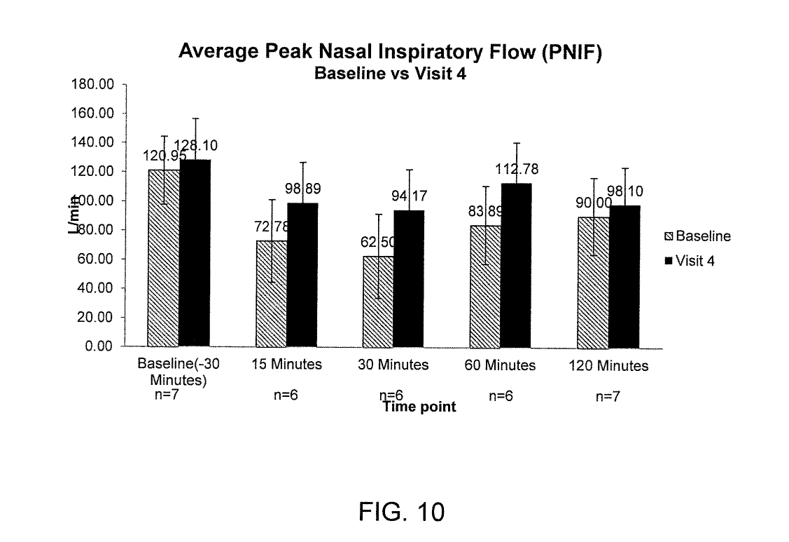

V4=31.94% (sig. 0.014). Significant at alpha=0.05. See FIG. 10.

V5=51.99% (sig. 0.009). Significant at alpha=0.01. See FIG. 11.

Note: Significance testing was performed using Paired t-test.

As shown in FIGS. 10 and 11, PNIF (L/min) post-challenge was improved dramatically in human subjects after treatment with Oxy Bump ‘T’ and Oxy Bump Eye Drops compared to baseline.

AUC nasal obstruction (VAS Nasal Symptom Score)—test of between subject effects of baseline vs. ARM 3 and ARM 4 (V4 and V5).

Percent Change (Other timepoint versus Baseline):

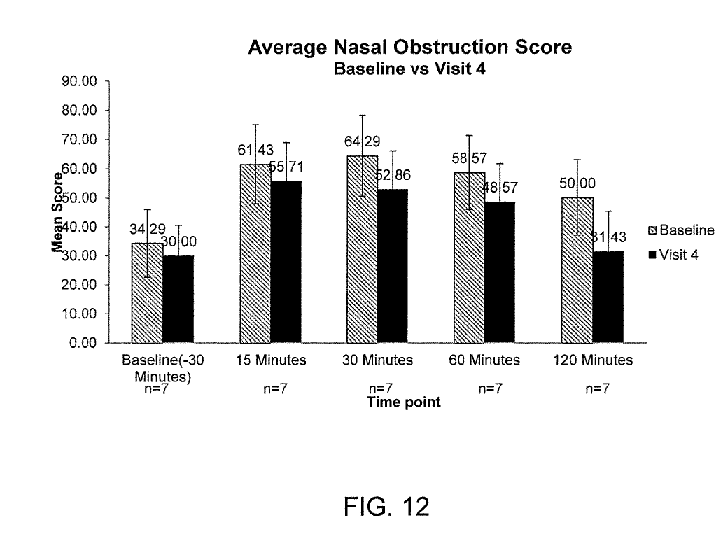

V4=−18.86% (sig. 0.174). See FIG. 12.

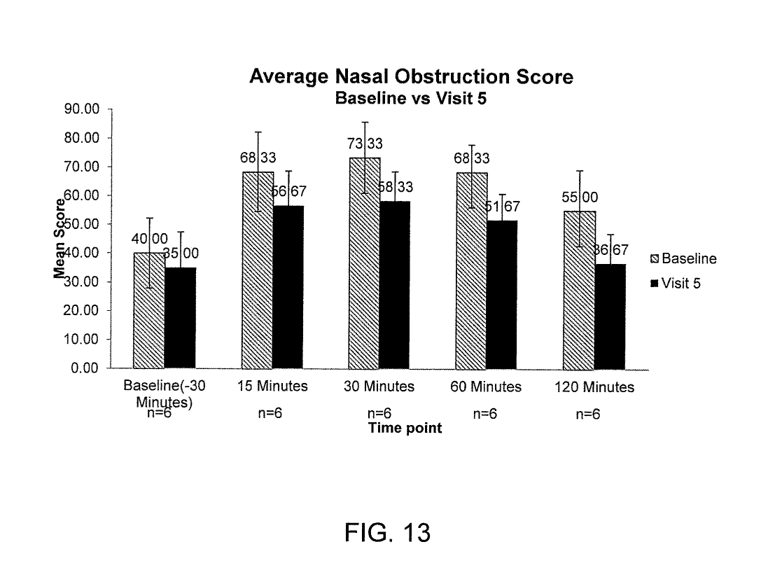

V5=−22.75% (sig. 0.012). See FIG. 13.

Note: Significance testing was performed using Paired t-test. Significant at alpha=0.05.

As shown in FIGS. 12 and 13, VAS nasal obstruction score post-challenge was improved dramatically after treatment with Oxy Bump ‘T’ and Oxy Bump Eye Drops compared to baseline.

AUC rhinorrhea (VAS Nasal Symptom Score)—test of between subject effects of baseline vs ARM 3 and ARM 4 (V4 and V5).

Percent Change (Other timepoint versus Baseline):

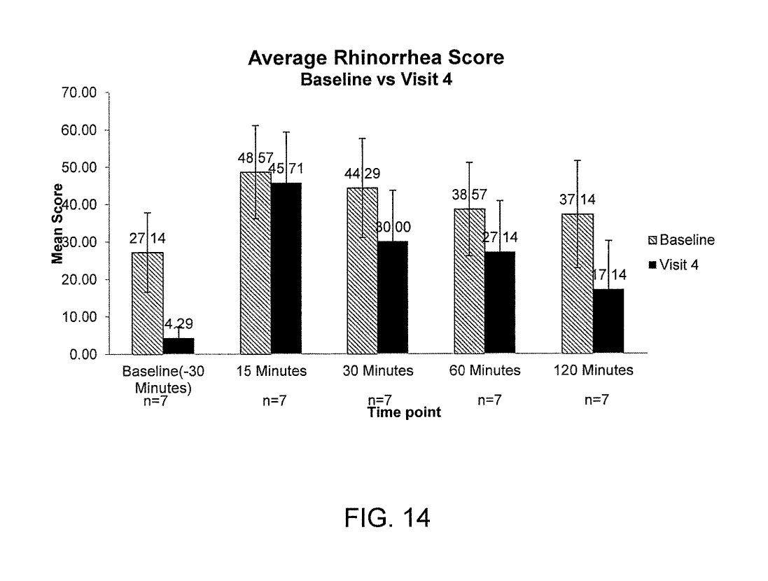

V4=−32.27% (sig. 0.098). See FIG. 14.

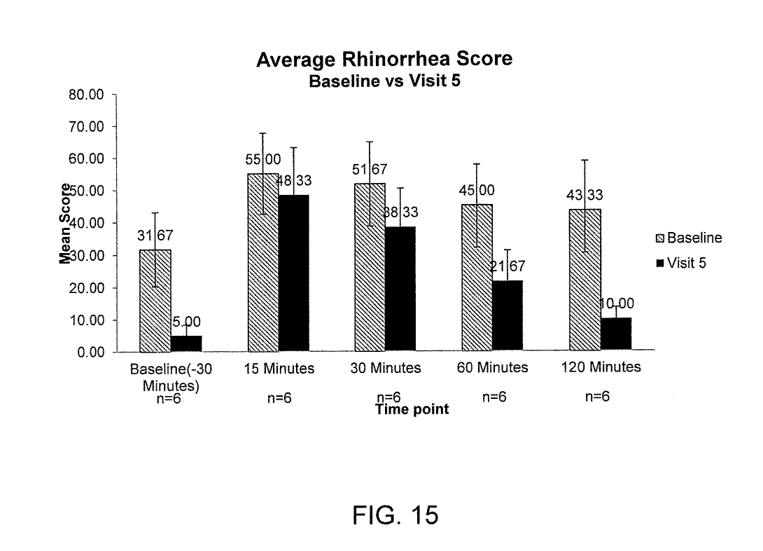

V5=−43.10% (sig. 0.003). Significant at alpha=0.01. See FIG. 15.

Note: Significance testing was performed using Paired t-test.

As shown in FIGS. 14 and 15, VAS rhinorrhea score post-challenge was improved dramatically after treatment with Oxy Bump ‘T’ and Oxy Bump Eye Drops compared to baseline.

Standardized Skin Scratch or Abrasion Protocol

Subject will undergo a standardized skin scratch protocol on both forearms. Subject will place both forearms evenly on the clean surface of a table. A ruler will be placed in between the forearms to serve as a frame of reference for measurement. Both forearms will be swabbed with an alcohol. Using a hypodermic needle, 3 parallel scratches will be made on the middle ⅓ of each forearm. The study product, Oxy Bump Skin spray, will be immediately administered to the scratches of one side.

Oxy Bump Skin Spray contains Stabilized Oxygen saline solution (AQUAGEN Maximum Performance), 50% by weight and balance water.

Subjects will be provided with the study product to take home and apply every 2 hours for a minimum of 6 applications. During each application, subject will place their arm on a level surface to apply product in 2-3 sprays with the goal of saturating the scratches completely. Subject will remain still to let Oxy Bump Skin Spray saturate skin for at least 60 seconds. In the next 24 hours, subjects will refrain from exposing scratches to excessive sweating and try to keep forearm area clean. A “FRIENDLY REMINDER” text will be sent at the appointed treatment times during the next 24 hours.

Subjects will return 24 hours later to take comparative pictures of the treated vs. untreated skin surfaces. Subject initials and Oxy Bump sticker will be placed on the subject's study product treated forearm. During the picture, the subject will place both forearms together so as to align the scratches on each arm next to each other.

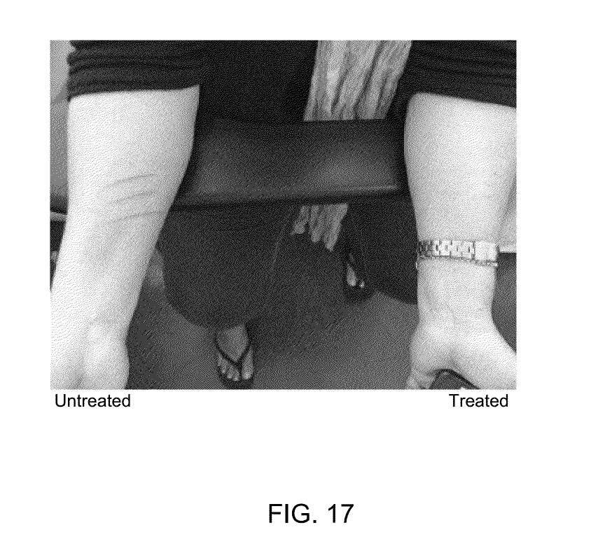

As shown in FIGS. 17-23, human subjects treated with the Oxy Bump Skin Spray showed a significant and dramatic improvement in the skin of the treated arm vs. the untreated arm after the abrasion testing.

FIG. 17 shows that the treated arm exhibited no scabbing, faint pink scratches, and flatter marks with no inflammation, while the untreated arm exhibited light scabbing, wider scratches having a faint red color surrounded by pink inflammation.

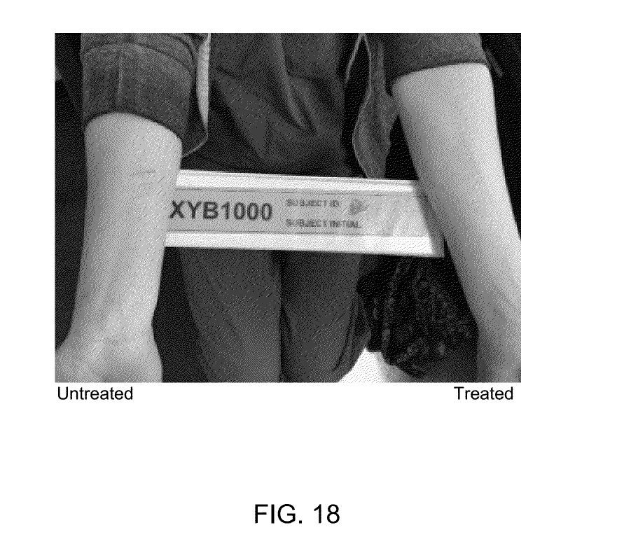

FIG. 18 shows that the treated arm exhibited no scabbing, faint marks hardly visible with no inflammation, while the untreated arm exhibited a top scratch with scabbing and wider diameter, bottom scratches having a light pink color.

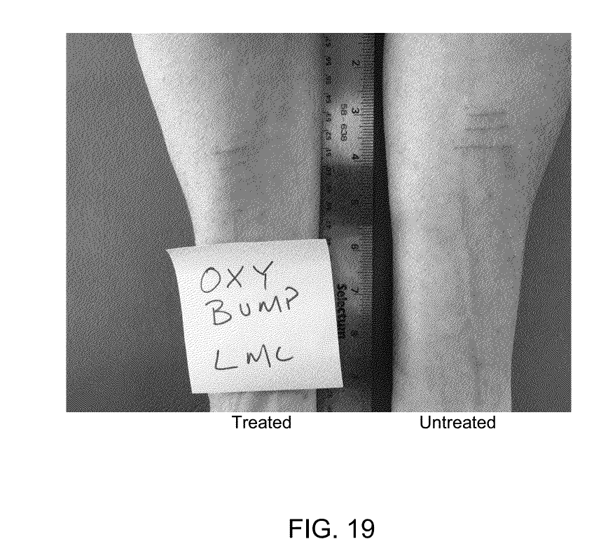

FIG. 19 shows that the treated arm exhibited top two scratches having light scabbing, bottom scratch pale pink with no inflammation, while the untreated arm exhibited light scabbing on all scratches having a light red color.

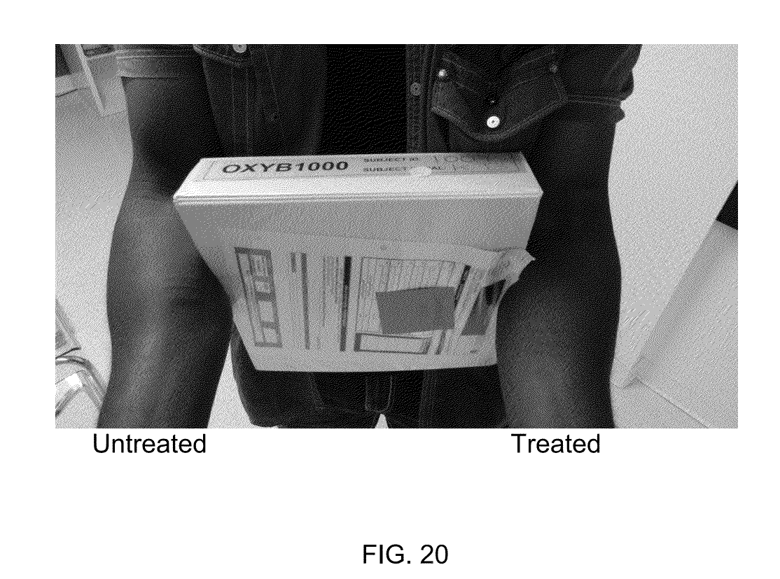

FIG. 20 shows that the treated arm exhibited flat scratches with no inflammation or scabbing, having a pale pink color, while the untreated arm exhibited light scabbing on all scratches with inflammation, having a light red color.

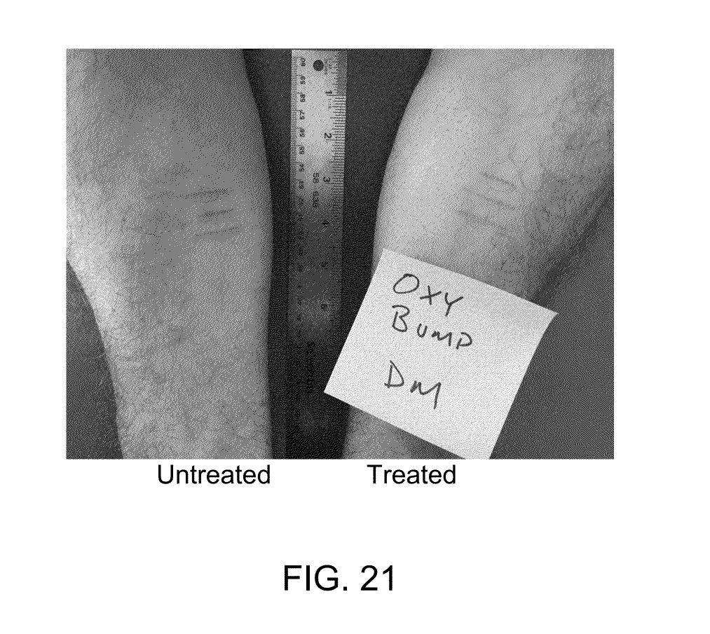

FIG. 21 shows that the treated arm exhibited a pink color, less inflammation and narrower scratch diameter, while the untreated arm exhibited scabbing, inflammation, wider diameter of the scratches, having a light red color.

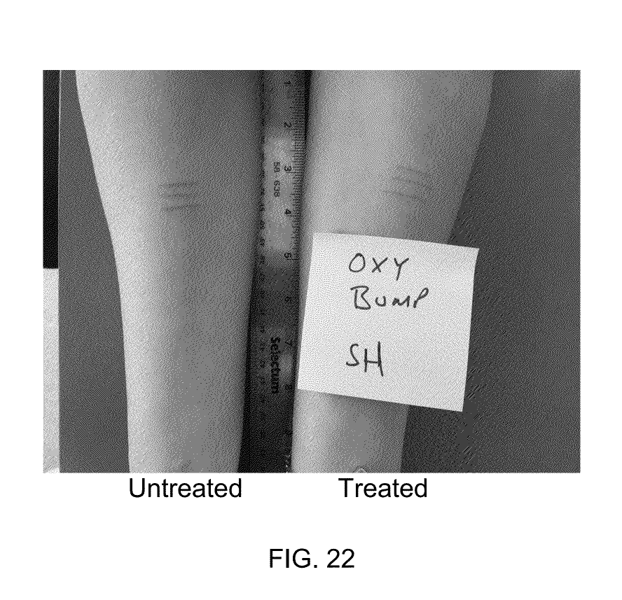

FIG. 22 shows that the treated arm exhibited light scabbing, having a pink color, and flatter scratches, while the untreated arm exhibited deeper scabbing, raised red scratches.

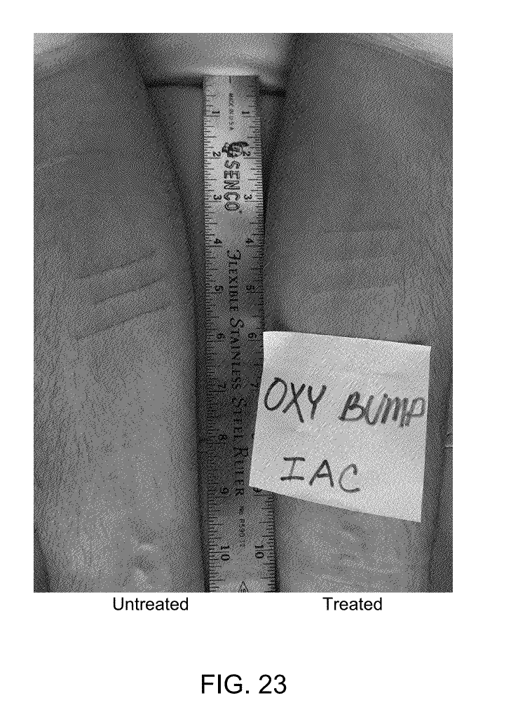

FIG. 23 shows that the treated arm exhibited a pink color, less inflammation and narrower scratch diameter, while the untreated arm exhibited scabbing, inflammation, wider diameter of the scratches, having a light red color.

For example, Oxy Bump ‘Skin Spray may be applied using 3-4 sprays on the surface of affected skin (e.g. on an area of from about 1 cm² skin surface area to about 10 cm² skin surface area) from a 30 ml bottle containing 240 sprays, 2×/day, or alternatively, as often as needed. Preferred dosage ranges for the skin spray can range about 0.2 g to about 1.0 g per day. Another suitable dosage range for the skin spray can range about 0.2 g to about 5.0 g per day.

Wounds are injuries that break the skin or other body tissues. They include cuts, scrapes, scratches, burns, electrical wounds, bites, stings, incisions, amputations, and punctured skin They often happen because of an accident, but surgery, sutures, and stitches also cause wounds.

As used herein, “wound healing” refers to a biologically-mediated process of repair or remodeling that may involve treatment of skin or other body tissues with a medicinal or nutritional composition. Such treatments as applied to the human body may include, but are not limited to, treatment of damage or insult to skin, tissue, and organs, wherein the affected area may be, for example, cutaneous, subcutaneous, visceral, circulatory, lymphatic, gastrointestinal, mucosal, skeletal, muscular, etc.

It has been shown that the oxygenated saline compositions containing stabilized oxygen, and optionally one or more homeopathic components, are useful in wound healing and skin repair.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A method for treating nasal congestion, allergy, throat, or eye in an individual, comprising the steps of:
(a) providing an oxygenated saline composition comprising stabilized oxygen and aqueous saline solution;
(b) administering to the individual in need thereof an effective amount of the oxygenated saline composition;
wherein the symptoms of nasal congestion, peak nasal inspiratory flow, allergy, throat irritation, or eye irritation are improved in the individual.

2. The method of claim 1, wherein the oxygenated saline composition further comprises at least one homeopathic component.

3. The method of claim 2, wherein the at least one homeopathic component is selected from the group consisting of Allium Cepa, Natrum Muriaticum, Histaminum Hydrochloricum, Quebracho, Luffa Operculata, Nux Vomica, Calcarea iodata, Euphrasia officinalis, Thuja occidentalis, Aconitum napellus, Ferrum phosphoricum, Hepar sulphuris calcareum, Kali bichromicum, Arnica Montana, Arum triphyllum, Belladonna, Bromium, Bryonia alba, Mercurius solubilis, Pulsatilla, Phytolacca decandra, Spongia tosta, Chamomilla, Sulphur, Calcarea Carbonica, Lycopodium, Adrenalinum, Alfalfa, Natrum Carbonic, Sarcolacticum Acidum, Lycopodium Clavatum, Lactic Acidum, Echinacea Purpurea, Baryta Carbonic, Lecithin, Carbo Vegetabilis, Kali iodatum, Thuja occidentalis, Apis mellifica, Cinchona officinalis, Graphites, Rhus toxicodendron, and mixtures thereof.

4. The method of claim 1, wherein the stabilized oxygen includes quad-atomic oxygen.

5. The method of claim 1, wherein the administering step is carried out using a nasal spray, and wherein the peak nasal inspiratory flow in the individual is increased by at least about 20%.

6. The method of claim 1, wherein the administering step is carried out using a nasal spray, and wherein the peak nasal inspiratory flow in the individual is increased by at least about 30%.

7. The method of claim 2, wherein the administering step is carried out using a nasal spray, and wherein the average peak nasal inspiratory flow in the individual is increased by at least about 24%.

8. The method of claim 1, wherein the administering step is carried out using a nasal spray, further wherein the nasal congestion is nasal obstruction or rhinorrhea, and further wherein nasal obstruction or rhinorrhea in the individual is decreased by at least about 20%.

9. The method of claim 2, wherein the administering step is carried out using a nasal spray, further wherein the nasal congestion is nasal obstruction or rhinorrhea, and further wherein nasal obstruction or rhinorrhea in the individual is decreased by at least about 20%.

10. The method of claim 1, wherein the administering step is carried out using a nasal spray, further wherein the eye irritation is itchy eyes, and further wherein itchy eyes in the individual is decreased by at least about 27%.

11. The method of claim 1, wherein the administering step is carried out using a nasal spray in an amount from about 0.2 g to about 2.0 g per day.

12. The method of claim 2, wherein the administering step is carried out using a nasal spray in an amount from about 0.2 g to about 2.0 g per day.

13. The method of claim 2, wherein the administering step is carried out using a throat spray, and wherein the peak nasal inspiratory flow in the individual is increased by at least about 30%.

14. The method of claim 13, wherein the administering step is carried out using the throat spray in an amount from about 0.2 g to about 2.0 g per day.

15. The method of claim 2, wherein the administering step is carried out using a throat spray, further wherein the nasal congestion is nasal obstruction or rhinorrhea, and further wherein nasal obstruction or rhinorrhea in the individual is decreased by at least about 30%.

16. The method of claim 15, wherein the administering step is carried out using the throat spray in an amount from about 0.2 g to about 2.0 g per day.

17. The method of claim 1, wherein the administering step is carried out using eye drops, and wherein the peak nasal inspiratory flow in the individual is increased by at least about 50%.

18. The method of claim 17, wherein the administering step is carried out using eye drops in an amount from about 0.1 g to about 0.6 g per day.

19. The method of claim 1, wherein the administering step is carried out using eye drops, further wherein the nasal congestion is nasal obstruction or rhinorrhea, and further wherein nasal obstruction or rhinorrhea in the individual is decreased by at least about 40%.

20. The method of claim 19, wherein the administering step is carried out using eye drops in an amount from about 0.1 g to about 0.6 g per day.

21. A method for treating damaged human skin or skin wounds in an individual, comprising the steps of:
(a) providing an oxygenated saline composition comprising stabilized oxygen and aqueous saline solution;
(b) administering to an individual in need thereof an effective amount of the oxygenated saline composition;
wherein the damaged skin is improved or healed in the individual.

22. The method of claim 21, wherein the skin wounds include cuts, scrapes, scratches, burns, electrical wounds, bites, stings, incisions, or punctures, and further wherein the skin wounds are substantially healed.

23. The method of claim 21, wherein the stabilized oxygen includes quad-atomic oxygen.

24. The method of claim 21, wherein the administering step is carried out using a spray or by directly swabbing or wiping onto the skin surface.

25. The method of claim 21, wherein the administering step is carried out in an amount from about 0.2 g to about 1.0 g per day when applied to a skin surface area of from about 1 cm² to about 10 cm².
